(19) United States
(12) Patent Application Publication
(10) Pub. No.: US 2009/0022818 A1
(43) Pub. Date: Jan. 22, 2009

(54) HIGH-FOAMING, VISCOUS CLEANSER COMPOSITION WITH A SKIN CARE AGENT

(75) Inventors: Ashoke K. SenGupta, Barrington, IL (US); Limin Liu, Palatine, IL (US); Gholam-Reza Vakili-Tahami, Naperville, IL (US); Ralph Spindler, Palatine, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: AMCOL International Corporation, Arlington Heights, IL (US)

(21) Appl. No.: 12/172,784

(22) Filed: Jul. 14, 2008

Related U.S. Application Data
(60) Provisional application No. 60/949,434, filed on Jul. 12, 2007.

(51) Int. Cl. A61K 31/60; C11D 3/16; A61K 33/04; A61K 31/05; A61K 31/07; A61K 31/135; A61K 31/7056; A61K 31/194; A61K 31/192; A61P 17/10

(52) U.S. Cl. 424/713; 510/137; 514/159; 514/734; 514/725; 514/646; 514/43; 514/574; 514/568

(57) ABSTRACT

A liquid cleansing composition comprising a cleansing surfactant, an adsorbed skin-care agent, adsorptive polymeric particles having an adsorption capacity of at least 2 g of skin care agent per gm of dry polymeric particles, a polymeric thickening agent having a molecular weight in the range of 500,000-10,000,000 Dalton, a water-soluble polymer having a molecular weight in the range of 200-50,000 Dalton, and a hydrophilic liquid, the said cleansing composition having the following features i) the low molecular weight, water-soluble polymer component functions as a foam-boosting and thixotropy-boosting agent; ii) the ratio of the Brookfield viscosity at 1 rpm of spindle speed to the Brookfield viscosity at 100 rpm of spindle speed is at least 10; and iii) the foam-volume is at least 20% higher with the water-soluble, polymeric, foam-boosting component than without it.

The present application claims the benefit of priority from U.S. provisional application Ser. No. 60/949,434, filed Jul. 12, 2007.

### BACKGROUND OF THE INVENTION

The present invention relates to high-foaming, liquid cleanser compositions. Specifically, it relates to high-foaming, facial cleanser compositions that contain as an active ingredient, for example, a skin-care active such as an anti-acne agent, namely, a combination of salicylic acid and benzoyl peroxide. More specifically, it relates to high-foaming, liquid cleanser compositions, wherein an amount of the active agent, e.g., anti-acne agent contained therein remains adsorbed (bound) onto the surface of water-insoluble, polymeric particles with a relatively high surface-area. The disclosed liquid cleanser compositions are sufficiently viscous, enabling the polymeric particles to remain stably suspended in these compositions. The claimed compositions are also highly shear-thinning, despite not containing any thixotropy-boosting ingredient known in the art, for which they can be used with pump dispensers. These cleanser compositions shun any possibilities of hetero-coagulation/flocculation (coagulation/flocculation of dissimilar particulate materials) of particulate ingredients contained therein.

For cleansing products, consumers often tend to equate the level of foaming to the cleansing power of these products, with the perception that, the higher the foaming, the better is the cleansing. Accordingly, in order to meet consumer acceptance, a cleanser preferably is capable of producing ample lather or suds, in the form of stable foam, when dispensed, for example, from a pump dispenser, as well as when rubbed against the skin during washing.

Furthermore, consumers prefer to use cleansing products that offer benefits beyond cleansing, for example, providing an active ingredient, such as a hair conditioning agent through a shampoo, or a fabric softening agent through a laundry detergent. In that vein, a facial cleanser, that contains an anti-acne agent, would be highly desirable from the standpoint of consumer preference. Typically, however, the anti-acne agents known in the art, such as a combination of salicylic acid and benzoyl peroxide, tend to cause skin-irritation, when the skin is exposed to relatively high levels of these materials. For a liquid cleanser, a way to avoid this skin-irritation problem is for the cleanser to contain the anti-acne agents in a form wherein an amount of the benefit agent is adsorbed onto the surface of a particulate material, preferably a particulate material having a relatively high surface-area.

AMCOL International Corporation produces and markets polymeric particle-based delivery systems for numerous active (benefit) agents. These polymeric particles are essentially crosspolymers of various types of comonomers, and are insoluble in water, hydrophilic organic solvents, and hydrophobic liquids. They typically present a relatively high surface-area, with their surfaces suitable for adsorbing benefit agents or active ingredients. Some of these polymeric particles can adsorb both hydrophobic and hydrophilic actives, having both hydrophobic and hydrophilic domains (areas) on the particle surface. One benefit of these particles is sustaining the delivery of the active over an extended period of time. Another benefit is improving the stability of actives loaded onto these particles. An added benefit of these microparticle delivery systems is that they help to control the skin-oil, while aiding the delivery of a skin-care agent to the skin.

Accordingly, the aforementioned polymeric particles can be used for binding onto their surfaces the foregoing anti-acne agents. Once “loaded” onto these particles, these active ingredients can be incorporated into a liquid cleanser formulation, wherein the chances for these actives to cause skin-irritation during washing would be greatly minimized. Nonetheless, in order to maintain these particles in suspension in a liquid cleanser composition, it is imperative that the viscosity of the composition is sufficiently high to prevent rapid settling of the particles during storage. Increasing the viscosity of a cleanser composition can be achieved by including thickening agents in the composition, as disclosed in U.S. Pat. No. 0,051,314 A1. However, dispensing a viscous cleanser through a foaming dispenser, for example, a pump foam dispenser, while maintaining good foaming, would be difficult, if not impossible, unless the composition was also highly shear-thinning or thixotropic. The invention presented in U.S. Pat. No. 0,051,314 A1 addressed this problem by combining a thixotropy-boosting agent, namely, a water-swellable clay, with water-soluble, polymeric thickening agents, wherein the liquid cleanser compositions disclosed therein exhibited high levels of thixotropy, and were found to be compatible with pump foam dispensers.

However, during the course of the research that led to the present invention, we found that the solution-approach revealed in U.S. Pat. No. 0,051,314 A1 could not be extended to the liquid cleanser compositions claimed herein. Adding a swellable clay to a cleanser composition of the present invention resulted in coagulation/flocculation of the particulate ingredients, namely, clay and polymeric particles. This undesirable outcome (coagulation/flocculation) of clay addition does not seem to be relevant for the compositions described in U.S. Pat. No. 0,051,314 A1, as the said disclosure does not provide any solution for addressing the problem, in anticipation of this outcome for the cleanser compositions described therein. However, in the context of the cleanser compositions claimed herein, the foregoing finding related to coagulation/flocculation seems reasonable upon considering the following mechanistic elements that are expected to come into play in driving coagulation/flocculation, with the addition of a swellable clay to the claimed compositions:
i) The pH of the cleanser compositions of the present invention can be strongly acidic (pH: 3.5-4.5), either due to the active agent, e.g., anti-acne agent, salicylic acid, contained therein, or that the stability of benzoyl peroxide can be increased in this pH range. When the pH is in the above range, portions (the edge-surface of plate-like particles/platelets of a water-swellable clay, for example, a smectite clay) of the clay-surface may provide a cationic surface charge, and/or an anionic charge on the face-surface of the clay platelets that may be screened out by the cationic component (hydrogen ion) of salicylic acid. A cationically-charged surface can undergo coagulation with an anionic surface and vice-versa. Also, a weakly charged surface (due to screening of surface charge) can coagulate with another surface due to the van der Waals attraction between the surfaces.
ii) The water-insoluble polymeric particles contained in the compositions of the present invention may adsorb any anionic or zwitterionic surfactant also contained in these compositions, due to which the surface charge of these particles may be either solely anionic or cationic or neutral (albeit with a combination of anionic and cationic charges on a zwitterionic surfactant molecule), depending on pH. In all these cases of surface charge, these polymeric particles may undergo coagulation with a swellable clay, especially when the pH of the compositions is strongly acidic, and/or when the surface of the polymeric particles is largely hydrophilic. An ionic surfactant (for example, an anionic surfactant) may impart a surface charge on a hydrophobic surface, upon adsorbing on the surface with the surfactant's hydrocarbon portion (as referred to in the art as surfactant “tail”) oriented towards the surface with the surfactant's charged group (as referred to in the art as surfactant “head”) facing outward from the surface. This surface charge could stabilize the suspended particles against coagulation. However, surfactants may not adsorb on a hydrophilic surface in the manner described above, for which a hydrophilic surface may not acquire a surface charge due to any adsorption of an ionic surfactant, which could potentially stabilize particle surfaces against coagulation. Some of the polymeric particles contained in the claimed compositions bear a considerable portion of hydrophilic domains on their surfaces.
iii) The claimed compositions may contain high molecular weight polymers as thickening agents. Any high molecular weight polymer may act as what is known as a bridging flocculant in the art.

Given all of the above, one object of the present invention is to produce high-foaming liquid cleanser compositions that contain an active ingredient, such as a skin-care agent, with an amount of the active (benefit) agent adsorbed onto the surface of certain water-insoluble polymeric particles, in order to minimize the chances of skin-irritation due to the active (benefit) agent during skin contact, e.g., washing. A further object is to have a sufficient viscosity (low-shear-rate viscosity) for the cleanser compositions, in order for maintaining good suspension of the polymeric particles, yet, while the compositions are highly shear-thinning for them to be usable with pump foam dispensers. A related object is to attain high-foaming and highly shear-thinning features for the claimed compositions, without the compositions containing a water-swellable clay, such as required in U.S. Pat. No. 0,051,314 A1, thus avoiding the risk of coagulation/flocculation of the particulate ingredients in the compositions, which can lead to undesirable separation of these ingredients during storage of the compositions. We have now found that the above object can be realized by having a water-soluble, polyether of a specific molecular weight and with no hydrophobic modification, as an ingredient in the claimed compositions. Nonetheless, if the molecular weight of the polyether exceeded a certain threshold value, the polymer then would result in coagulation/flocculation of the polymeric particles in the claimed compositions. The unexpected nature of this finding lies in polyether polymers of the type noted above, not being disclosed in the prior art as additives for boosting foaming and shear-thinning properties of cleansing compositions.

The known methods for boosting foaming of cleansing compositions include:
i) Use of foaming agents including foaming surfactants, and in particular, amphoteric surfactants, for example, cocamidopropyl betaine; and
ii) Use of fatty alcohols (for example, lauryl alcohol), and fatty amides (for example, cocomonoethanolamide)

However, because the cleanser compositions of the present invention are highly shear-thinning, the fatty alcohol- and fatty amide-based foam-boosters are not suitable, since these additives tend to increase the viscosity, while reducing the shear-thinning property of cleanser compositions. Also, even though the claimed compositions may include cocamidopropyl betaine as an amphoteric cleansing surfactant, we have now found a novel method for boosting foaming of these compositions, over and above that due to a foaming agent such as an amphoteric surfactant. Equally unexpected, the method is also found to increase the shear-thinning property of the cleanser compositions claimed herein.

### SUMMARY OF THE INVENTION

The compositions claimed in the present invention represent a liquid cleanser, particularly useful for cleansing the facial skin. The salient composition and beneficial features of these compositions are given below.
High-foaming, having an excellent foam stability
Viscous, having a Brookfield viscosity of about 1,000-50,000 cps at 1 rpm of spindle speed, while the composition comprises a thickening agent that is not based on any derivatives of high molecular weight polyethylene glycol, fatty acids, fatty alcohols, fatty esters, and salts of fatty acids
Yet, sufficiently shear-thinning, wherein the ratio (referred to herein as shear-thinning index) of Brookfield viscosity at 1 rpm of spindle speed to Brookfield viscosity at 100 rpm of spindle speed is at least 10, more preferably at least 25, and most preferably at least 40, such that the composition can be dispensed easily through a pump dispenser or such similar devices
Contains about 1-50% by weight of detersive surfactants selected from anionic, nonionic, zwitterionic, and cationic surfactants
Contains an active ingredient, such as a skin-care agent in an amount of 0.01-20% by weight, wherein an amount of the active (benefit) agent remains adsorbed onto the surface of water-insoluble polymeric particles having a relatively high surface-area
More specifically, in one embodiment, the active skin-care agent is an anti-acne agent selected from salicylic acid, benzoyl peroxide, sulfur, resorcinol, tretinoin, adapolene, dapsone, benzamyacin, and clindomyacin, or combinations thereof.
Contains a polymeric or copolymeric thickening agent selected from the group consisting of high molecular weight (molecular weight>500,000 Dalton) gum polymers, cellulosic polymers, polyacrylate, polyacrylamide, including cross-polymers and hydrophobically-modified polymers, at an amount of 0.1-10% by weight
Contains a low molecular weight, water-soluble polymer selected from the group consisting of polyether, polycarboxylate, polyphosphate, polyphenol, polyamine, and polyamide having a molecular weight in the range of 200-50,000 Dalton, at an amount of 0.1-20% by weight

### DETAILED DESCRIPTION OF THE INVENTION

The present invention discloses high-foaming, liquid cleanser compositions, particularly suited for cleansing the facial skin, because of a skin-care agent contained therein. These cleanser compositions embody a feature for minimizing any skin-irritation potential due to the skin-care agent, by having an amount of the skin-care agent remaining adsorbed on the surface of certain water-insoluble polymeric particles, with the polymeric particles stably suspended in the claimed compositions. High molecular weight polymeric thickening agents are used in order to render these compositions sufficiently viscous for maintaining good suspension of the polymeric particles. These cleanser compositions are highly shear-thinning, apparently due to the inclusion of a low molecular weight, water-soluble polymer as an ingredient, which is also found to enhance the foaming ability of these compositions, beyond what can be attributed to the foam-producing, cleansing surfactants contained therein. The polymer preferably is not a hydrophobically-modified and/or a cross-linked polymer, and if it is an anionic polymer and/or a copolymer, it is preferably free of any strong acid groups (for example, sulfate group or sulfonate group). These cleansing surfactants are selected from anionic, nonionic, zwitterionic, and cationic surfactants, which remain dissolved in a hydrophilic liquid base of the claimed compositions. The liquid base is primarily composed of water.

The various aspects of the aforementioned compositions are discussed in a greater detail below:

### Surfactants

Non-limiting examples of suitable anionic surfactants are the sodium, ammonium, and mono-, di-, and tri-ethanolamine salts of alkyl sulfates, alkyl ether sulfates, alkyl sulfonates, alkaryl sulfonates, alkyl succinates, alkyl sulfosuccinate, N-alkoyl sarcosinates, alkyl phosphates, alkyl ether phosphates, alkyl ether carboxylates, and α-olefin sulfonates. The alkyl groups generally contain from 8 to 18 carbon atoms and may be unsaturated. The alkyl ether sulfates, alkyl ether phosphates, and alkyl ether carboxylates may contain from about 1 to about 10 ethylene oxide or propylene oxide units per molecule, and preferably contain 2 to 3 ethylene oxide units per molecule. Examples of the most preferred anionic surfactants include sodium or ammonium olefin sulfonate, sodium or ammonium lauryl sulfate and sodium or ammonium lauryl ether sulfate.

Suitable nonionic surfactants include, but not limited to, aliphatic, primary or secondary linear or branched chain alcohols or phenols with alkylene oxides, generally ethylene oxide and generally 6-30 ethylene oxide groups. Other suitable nonionic surfactants include mono- or di-alkyl alkanolamides, alkyl polyglucosides, and polyhydroxy fatty acid amides.

The zwitterionic/amphoteric surfactants suitable for use in the present invention include alkyl amine oxides, alkyl betaines, alkyl amidopropyl betaines, alkyl sulfobetaines, alkyl glycinates, alkyl carboxyglycinates, alkyl amphopropionates, alkyl amidopropyl hydroxysultaines, acyl taurates, and acyl glutamates wherein the alkyl and acyl groups have from 8 to 18 carbon atoms.

Nonlimiting examples of suitable cationic surfactants include water-soluble or water-dispersible or water-insoluble compounds containing at least one amine group which is preferably a quaternary amine group, and at least one hydrocarbon group which is preferably a long-chain hydrocarbon group. The hydrocarbon group may be hydroxylated and/or alkoxylated and may comprise ester- and/or amido- and/or aromatic-groups. The hydrocarbon group may be fully saturated or unsaturated.

The level of surfactants may range from 1 to 50%, preferably from 2 to 30%, and most preferably from 3 to 20% by weight of the claimed compositions.

### Hydrophilic Liquids

The hydrophilic liquids suitable for use include water and hydrophilic organic liquids and mixtures thereof. Nonlimiting examples of preferred hydrophilic organic liquids include glycerol, ethanol, isopropanol, propylene glycol, butylene glycol, and hexylene glycol, and mixtures thereof.

The level of hydrophilic liquids may range from 50 to 95%, preferably from 60 to 90%, and most preferably from 70 to 90% by weight of the claimed compositions.

### Skin-Care Agents

Nonlimiting examples of skin-care agents suitable for the present invention include salicylic acid, glycolic acid, ascorbic acid or its derivatives, benzoyl peroxide, retinol or its derivatives, adapolene, sulfur, resorcinol, clindamyacin, benzamyacin, azelaic acid, peptides, proteins, emollients, tocopherol, oil-soluble sunscreens, and tretinoin. The amount of the skin-care agents contained in the claimed compositions may range from 0.01-20% by weight of the composition.

### Polymeric Particles for Binding the Anti-Acne Agents

Several different types of adsorbent polymeric microparticles may be suitable for the object of the present invention. One class of adsorbent polymeric microparticles is prepared by suspension polymerization techniques, as set forth in U.S. Pat. Nos. 5,677,407; 5,712,358; 5,777,054; 5,830,967; 5,834,577; 5,955,552; and 6,107,429, each incorporated herein by reference (available commercially under the tradename of POLY-PORE® E200, INCI name, allyl methacrylate crosspolymer, from AMCOL International, Arlington Heights, Ill.). Another class of adsorbent polymeric microparticles is prepared by a precipitation polymerization technique, as set forth in U.S. Pat. Nos. 5,830,960; 5,837,790; 6,248,849; and 6,387,995, each incorporated herein by reference (available commercially under the tradename of POLY-PORE® L200 from AMCOL International, Arlington Heights, Ill.).

Another class of adsorbent polymeric microparticles prepared by a precipitation polymerization technique is disclosed in U.S. Pat. Nos. 4,962,170; 4,948,818; and 4,962,133, each incorporated herein by reference, and available commercially under the tradename of POLYTRAP by AMCOL International (INCI name of lauryl methacrylate/glycol dimethacrylate crosspolymer).

An additional adsorbent polymeric microparticle has been developed, as disclosed in U.S. Pat. Re. 33,429, incorporated herein by reference and marketed under the tradename of MACROBEAD by AMCOL International (INCI name of lauryl methacrylate/glycol dimethacrylate crosspolymer). Other adsorbent polymers that are commercially available include, for example, MICROSPONGE® (INCI name of methyl methacrylate/glycol dimethylacrylate crosspolymer), as disclosed in U.S. Pat. No. 4,690,825, available from AMCOL International, and Poly-HIPE polymers (e.g., a copolymer of 2-ethylhexyl acrylate, styrene, and divinylbenzene) available from Biopore Corporation, Mountain View, Calif.

The amount of the skin-care agent loaded onto the polymeric microparticle may range from about 1% to about 85% of the total weight of skin-care agent-polymeric microparticle system. The maximum adsorption capacity of the polymeric microparticles for either hydrophobic or hydrophilic skin care agents may be in the range of about 2-15 g of adsorbate/gm of the dry polymer.

### Polymeric Thickening Agents

Various types of high molecular weight polymeric thickening agents may be used in the compositions disclosed herein. These polymers have a molecular weight in the range of 500,000-10,000,000 Dalton. They may be homopolymers or copolymers. They may be cross-linked polymers or hydrophobically-modified polymers. The preferred polymeric thickening agents for the object of the present invention include the various gum polymers, cellulosic polymers, acrylate-based polymers, methacrylate-based polymers, and acrylamide-based polymers. Examples of the most preferred polymeric thickening agents for the claimed cleansing compositions include polyacrylate-1 (Carbopol® Aqua CC from Lubrizol), xanthan gum, cationic guar gum, and ammonium acryloyldimethyltaurate/beheneth-25 methacrylate crosspolymer. The amount of the polymeric thickening agent in the cleansing compositions may vary from 0.05 to 5%, preferably from 0.1 to 3%, and most preferably from 0.5-2% by weight of the compositions.

### Thixotropy- and Foam-boosting Agent

The prior art teaches that polymer-surfactant complexes may be produced in compositions comprising water-soluble, polymeric thickening agents and water-soluble surfactants, due to electrostatic (for example, complexes formed between anionic surfactants and cationic polymers), hydrogen-bonding, and hydrophobic interactions. Depending on the hydrophilic-lipophilic balance (HLB) features of polymers and surfactants mixed in a given water-based composition, a polymer-surfactant complex formed therein can be more surface-active (wherein surface-activity is defined as the ability of a material to adsorb at an air-water interface) than the polymer or the surfactant comprising the complex. The increased surface-activity, coupled with a polymer's innate ability to stabilize interfaces upon interfacial adsorption, due to mechanisms described in the colloid literature, may render such a polymer-surfactant complex capable of functioning as a strong foam-stabilizer (involving stabilization of air-water interface).

Conversely, depending on the HLB features of its components, the polymer-surfactant complex can work against foam-stabilization. In particular, for example, a complex formed between a hydrophobically-modified cationic polymer and an anionic surfactant can adversely affect foaming, due to an increased lipophilicity of the complex. However, the same complex, after undergoing further complexation (for example, through hydrogen-bonding), for example, with a second polymer, can turn into a strong foam-stabilizer, provided that the HLB feature for the resulting complex is suited for imparting a relatively high level of foam-stabilization.

Without an effective foam-boosting agent contained therein, the cleanser compositions of the present invention could present problems towards attaining good foaming, due to polymer-surfactant complexes that could potentially form in these compositions. Nonetheless, especially, given the need for these compositions to be highly shear-thinning, the foam-boosters known in the art would not be suitable for the object of the present invention, as noted in an earlier section.

It has now been found unexpectedly that certain water-soluble oligomers or polymers of ethylene oxide, when included as an ingredient, can render the cleanser compositions of the present invention high-foaming and highly shear-thinning. For the object of the present invention, these polyether (having ether oxygen-groups along the polymer chain) materials are required to be homopolymers having a molecular weight in the range of 200-10,000 Dalton. The most preferred polyether is a polyethylene glycol polymer which has not been copolymerized and/or hydrophobically-modified to have lipid- or oil-like properties. A polyethylene glycol polymer having a molecular weight exceeding 10,000 Dalton cannot be used in the compositions of the present invention, as it may result in coagulation of the polymeric particles contained in these compositions. Also, any polyether or polyethylene glycol polymer (wax) that can only be dispersed or suspended throughout the claimed compositions as visible droplets or particles, by not being completely soluble in the said compositions, is not usable in the present invention. The amount of the preferred, water-soluble, foam- and thixotropy-boosting agent, in the cleansing compositions may vary from 0.05 to 10%, preferably from 0.5 to 7.5%, and most preferably from 1-5% by weight of the compositions.

Nonlimiting examples of additional materials to function as a foam-booster in the present compositions may include low molecular weight (molecular weight in the range of 1,000-50,000 Dalton, preferably in the range of 2,000-25,000 Dalton, and most preferably in the range of 5,000-10,000 Dalton) polymers and copolymers selected from the group consisting of polycarboxylate (especially suitable in the pH range of 3.5-4.5), polyphosphate (especially suitable in the pH range of less than 6.5), polyphenol, polyamine, polyamide. If the foam-booster is a copolymer, it is preferably free of any hydrophobic comonomer and/or a hydrophilic comonomer with a strong acid group (for example, sulfate group or sulfonate group), and is preferably not a crosspolymer. The foam-boosting polymer suitable for the present invention is also completely soluble in water, and not just dispersible in water. It is preferably not hydrophobically-modified, nor does it function as a surfactant or a foaming agent.

### Optional Ingredients

Nonlimiting examples of the optional ingredients that may included as ingredients in the cleanser compositions of the present invention includes dyes, preservatives, fragrances, plant or botanical extracts, antioxidants, vitamins, humectants, water-soluble sunscreens, and chelating agents.

In order to further illustrate the present invention, the following nonlimiting examples are presented. However, they should not be construed as limiting the scope of the invention to their details.

### EXAMPLE I

This example shows some typical compositions of the present invention, which contain salicylic acid as a skin-care agent. The addition of PEG-8 greatly improved the foaming ability of the two formulations presented in Table I.

TABLE I

| | Formula- | Formula- |
|---|---|---|
| | tion 1, | tion 2, |
| Ingredient | Weight % | Weight % |
| Deionized Water | 44.18 | 50.77 |
| Polyacrylate-1 Crosspolymer, 20 [symbol] | 7.00 | 0.00 |
| active, (Carbopol ® Aqua CC fro [symbol] | | |
| Lubrizol) | | |
| Xanthan Gum | 0.00 | 0.50 |
| Polyethylene Glycol (PEG-8) | 4.20 | 3.6 |
| Sodium C₁₄₋₁₆ Olefin Sulfonate, 40 [symbol] | 28.00 | 28.00 |
| active | | |
| Cocamidopropyl Betaine, 30% active | 3.40 | 3.40 |
| 11.16% (w/w) Dispersion in Water [symbol] | 8.96 | 8.96 |
| Allyl Dimethacrylate Crosspolym [symbol] | | |
| loaded with Salicylic Acid (PolyPor [symbol] | | |
| 150SA from AMCOL) | | |
| Salicylic Acid | 1.50 | 1.50 |
| Butylene Glycol | 0.80 | 0.80 |
| Glycerin | 0.20 | 0.20 |
| Tetrasodium EDTA | 0.10 | 0.10 |
| 20% (w/w) Sodium Hydroxide | 0.50 | 1.00 |
| Vitamin E Acetate | 0.10 | 0.10 |
| Licorice extract | 0.10 | 0.10 |
| Witch Hazel Extract | 0.10 | 0.10 |
| Calendula Extract | 0.10 | 0.10 |
| Sodium Benzotriazolyl Butylphen [symbol] | 0.03 | 0.03 |
| Sulfonate (Tinogard ® HS, Ciba Special [symbol] | | |
| Chemicals) | | |
| Preservative, Fragrance, Color | 0.73 | 0.74 |

[symbol]

indicates data missing or illegible when filed

### EXAMPLE II

This example demonstrates the boosting of the shear-thinning property of the compositions of the present invention, due to PEG-8. In order to determine the effect, corresponding to each of polyacrylate-1 and xanthan gum as a polymeric thickening agent, formulations were prepared akin to the ones presented in Table II, wherein water was used to replace PEG-8 in the formulations that did not contain any PEG-8. Also, in each formulation, the entire amount of salicylic acid was added in its native form (i.e., none of it was adsorbed onto the microparticle Polytrap®). This enabled determining separately (using formulations without the microparticle, akin to the ones in Table II) that the microparticle by itself did not have any profound effect on the shear-thinning property.

TABLE II

| | Formula- | Formula- |
|---|---|---|
| | tion 1, | tion 2, |
| Ingredient | Weight % | Weight % |
| Deionized Water | 43.90 | 50.27 |
| Polyacrylate-1 Crosspolymer, 20% active, | 7.14 | 0.00 |
| (Carbopol ® Aqua CC from Lubrizol) | | |
| Xanthan Gum | 0.00 | 0.50 |
| Polyethylene Glycol (PEG-8) | 4.20 | 3.6 |
| Sodium C14-16 Olefin Sulfonate, 40% | 28.00 | 28.00 |
| active | | |
| Cocamidopropyl Betaine, 30% active | 3.40 | 3.40 |
| 5.574% (w/w) Dispersion in Water of | 8.97 | 8.97 |
| Lauryl Methacrylate/Glycol | | |
| Dimethacrylate Crosspolymer | | |
| (Polytrap ® from AMCOL) | | |
| Salicylic Acid | 2.00 | 2.00 |
| Butylene Glycol | 0.80 | 0.80 |
| Glycerin | 0.20 | 0.20 |
| Tetrasodium EDTA | 0.10 | 0.10 |
| 20% (w/w) Sodium Hydroxide | 0.20 | 1.00 |
| Vitamin E Acetate | 0.10 | 0.10 |
| Licorice extract | 0.10 | 0.10 |
| Witch Hazel Extract | 0.10 | 0.10 |
| Calendula Extract | 0.10 | 0.10 |
| Sodium Benzotriazolyl Butylphenol | 0.03 | 0.03 |
| Sulfonate (Tinogard ® HS, Ciba Specialty | | |
| Chemicals) | | |
| Preservative, Fragrance, Color | 0.66 | 0.73 |

The following table shows the increase in the shear-thinning index (the ratio of Brookfield viscosity at 1 rpm of spindle speed to the Brookfield viscosity at 100 rpm of spindle speed), due to the addition of PEG-8 in the claimed compositions.

TABLE III

| | Brookfield Viscosity, | | | | Brookfield Viscosity, | | |
|---|---|---|---|---|---|---|---|
| Brookfield | cps, with Polyacrylate-1 | | | | cps, with Xanthan Gum | | |
| Viscometer | No PEG-8 | | No | With | No PEG-8 | | With |
| Spindle | and No | | Polytrap, | Polytrap | and No | | Polytrap |
| Speed, rpm | Polytrap | No PEG-8 | with PEG-8 | and PEG-8 | Polytrap | No PEG-8 | and PEG-8 |
| 1 | 20400 | 22000 | 15200 | 22000 | 13600 | 12400 | 13200 |
| 2 | 11600 | 12200 | 8400 | 11800 | 7400 | 6800 | 7200 |
| 4 | 6800 | 6900 | 4900 | 6700 | 4100 | 4400 | 4000 |
| 5 | 5440 | 5840 | 4080 | 5240 | 3360 | 3200 | 3360 |
| 10 | 3280 | 3480 | 2320 | 3120 | 1880 | 1800 | 1880 |
| 20 | 2120 | 2200 | 1380 | 1740 | 1100 | 1040 | 1060 |
| 50 | 1256 | 1280 | 704 | 888 | 544 | 512 | 520 |
| 100 | 908 | 912 | 432 | 568 | 320 | 308 | 304 |
| Shear-thinning | 22.47 | 24.12 | 35.19 | 38.73 | 42.50 | 40.26 | 43.42 |
| Index | | | | | | | |

### EXAMPLE III

This example shows the foam-boosting effect of PEG-8 in the compositions of the present invention. The formulations used in conducting the relevant testing of foam-volume, using a test method known in the art, are akin to the formulations in Table II.

TABLE IV

| Foam-volume, mL | | | | Foam-volume, mL | | | Foam-volume, mL | | |
|---|---|---|---|---|---|---|---|---|---|
| Polymeric Thickener: | | | | Polymeric Thickener: | | | Polymeric Thickener: | | |
| Polyacrylate-1 | | | | Xanthan Gum | | | Cationic Guar Gum | | |
| No PEG-8, | No | No Polytrap | With PolyTrap | No PEG-8, | No | With Polytrap | No PEG-8, | No | With Polytrap |
| No Polytrap | PEG-8 | with PEG-8 | and PEG-8 | No Polytrap | PEG-8 | and PEG-8 | No Polytrap | PEG-8 | and PEG-8 |
| 2.6 | 3.6 | 5.6 | 5.0 | 6.7 | 7.2 | 9.0 | 4.8 | 4.5 | 6.2 |

### EXAMPLE IV

This example presents a high-foaming composition of the present invention, wherein the skin-care agent is benzoyl peroxide loaded onto to Poly-Pore®.

TABLE V

| Ingredient | Weight % |
|---|---|
| Deionized Water | 51.28 |
| Xanthan Gum | 0.70 |
| Polyethylene Glycol (PEG-8) | 3.60 |
| Sodium C14-16 Olefin Sulfonate, 40% active | 28.00 |
| Cocamidopropyl Betaine, 30% active | 3.40 |
| Allyl Methacrylate Crosspolymer loaded with Benzoyl | 6.60 |
| Peroxide (Poly-Pore ® 438BP from AMCOL) | |
| Glycerin | 5.00 |
| Tetrasodium EDTA | 0.10 |
| 50% (w/w) Citric Acid | 0.72 |
| Sodium Citrate | 0.30 |
| Preservative | 0.30 |

What is claimed is:

1. A liquid cleansing composition comprising a cleansing surfactant, an adsorbed skin-care agent, adsorptive polymeric particles having an adsorption capacity of at least 2 g of skin care agent per gm of dry polymeric particles, a polymeric thickening agent having a molecular weight in the range of 500,000-10,000,000 Dalton, a water-soluble polymer having a molecular weight in the range of 200-50,000 Dalton, and a hydrophilic liquid, the said cleansing composition having the following features:
i) the low molecular weight, water-soluble polymer component functions as a foam-boosting and thixotropy-boosting agent;
ii) the ratio of the Brookfield viscosity at 1 rpm of spindle speed to the Brookfield viscosity at 100 rpm of spindle speed is at least 10; and
iii) the foam-volume is at least 20% higher with the water-soluble, polymeric, foam-boosting component than without it.

2. The liquid cleansing composition of claim 1, wherein:
i) the composition is free of any particulate thixotropic agent; and
ii) the adsorptive polymeric particles remain free of heterocoagulation with any dissimilar particulate components.

3. The composition of claim 1 wherein the cleansing surfactant is selected from the group consisting of anionic surfactants, non-ionic surfactants, zwitterionic surfactants, cationic surfactants, and mixtures thereof.

4. The composition of claim 3 wherein the amount of the cleansing surfactant is about 1% to about 50% by weight of the composition.

5. The composition of claim 1 wherein the skin-care agent is an anti-acne agent selected from the group consisting of salicylic acid, sulfur, resorcinol, tretinoin, adopolene, dapsone, clindamyacin, benzamyacin, azelaic acid, benzoyl peroxide, and combinations thereof.

6. The composition of claim 5 wherein the amount of the anti-acne agent is about 0.01% to about 20% by weight of the composition.

7. The composition of claim 1 wherein the adsorptive polymeric particle is selected from the group consisting of allyl methacrylate crosspolymer, lauryl methacrylate/glycol dimethacrylate crosspolymer, and methyl methacrylate/glycol dimethacrylate crosspolymer.

8. The composition of claim 7 wherein the amount of the adsorptive polymeric particle is about 0.01% to about 10% by weight of the composition.

9. The composition of claim 1 wherein the polymeric thickening agent is a polymer or a copolymer selected from the group consisting of gum polymers, cellulosic polymers, acrylate polymers, acrylamide polymers, and mixtures thereof.

10. The composition of claim 9 wherein the amount of the polymeric thickening agent is about 0.05% to about 5% by weight of the composition.

11. The composition of claim 1 wherein the water-soluble polymer, functioning as a foam-boosting and thixotropy-boosting agent, is selected from the group consisting of polyether, polycarboxylate, polyphosphate, polyphenol, polyamine, and polyamide.

12. The composition of claim 11 wherein the water-soluble polymer is polyether.

13. The composition of claim 12 wherein the polyether is a homopolymer, and is not hydrophobically-modified or cross-linked.

14. The composition of claim 13 wherein the polyether has a molecular weight in the range of 200-6,000 Dalton.

15. The composition of claim 14 wherein the polyether is polyethylene glycol.

16. The composition of claim 11 wherein the amount of the water-soluble polymer is about 0.1% to about 10% by weight of the composition.

17. A method of increasing the shear-thinning and foaming properties of the composition of claim 1 by dissolving a water-soluble polyether polymer having a molecular weight in the range of 200-6,000 Dalton in the composition in an amount of about 0.1% to about 10% by weight of the composition.

18. The composition of claim 5, wherein the skin care agent is selected from the group consisting of salicylic acid and benzoyl peroxide.
